(19) United States
(12) Patent Application Publication
(10) Pub. No.: US 2023/0335297 A1
(43) Pub. Date: Oct. 19, 2023

(54) Pathogen Clearance System and Method

(71) Applicant: Bayer HealthCare LLC, Whippany, NJ (US)

(72) Inventors: Shyam PANJWANI, Fremont, CA (US); Konstantinos SPETSIERIS, San Francisco, CA (US); Michal MLECZKO, Sacramento, CA (US); Wensheng WANG, Pittsburg, CA (US); June Zou, Richmond, CA (US); Mohammad ANWARUZZAMAN, Hercules, CA (US); Oliver HESSE, Berkeley, CA (US); Roger CANALES, Berkeley, CA (US); JIARONG CUI, Newark, CA (US); Shengjiang LIU, Lafayette, CA (US)

(21) Appl. No.: 18/044,132

(22) PCT Filed: Aug. 23, 2021
(86) PCT No.: PCT/US2021/047048

§ 371 Date: Mar. 6, 2023

Related U.S. Application Data
(60) Provisional application No. 63/080,856, filed on Sep. 21, 2020.

(51) Int. Cl. G16H 50/80; G16H 50/20

(52) U.S. Cl. CPC G16H 50/80; G16H 50/20

(57) ABSTRACT

The present embodiments relate to pathogen clearance. Subject matter of the present embodiments are computer-implemented methods, computer systems and computer-readable storage media for predicting the performance of pathogen clearance processes.

### CROSS-REFERENCE TO RELATED APPLICATIONS

This application claims the benefit of U.S. Provisional Patent Application No. 63/080,856, filed on Sep. 21, 2020, the disclosure of which is hereby incorporated by reference in its entirety.

### BACKGROUND

Today, the majority of recombinant therapeutic proteins are manufactured by large-scale fermentation of animal or human derived host cells that are genetically engineered to express the genes of interest. Typical host cells include the baby hamster kidney (BHK-21), Chinese hamster ovarian (CHO), mouse myeloma (NSO), and some potential human cell lines i.e. HEK293.

The production of recombinant therapeutic proteins from animal or human cell lines entails the risk of contaminations by pathogens.

Host cells may contain endogenous retrovirus-like particles (ERVLPs) coding sequences (provirus like) intrinsically integrated in their chromosomes. ERVLPs are spontaneously produced in cell cultures and cause viral contamination.

Adventitious agents could be introduced into the bioprocess or the final product via cell substrates, raw materials and mechanical, environmental, personnel and process-related factors.

Therefore, effective pathogen clearance such as viral removal and/or inactivation by the manufacturing process are crucial to ensure pathogen safety of biologicals.

Pathogen clearance is usually achieved by dedicated unit operations, such as low pH inactivation, viral filtration, chromatographic separation and/other techniques.

The capacity and robustness of the pathogen clearance by a manufacturing process must be validated by viral clearance studies using a scale-down model. The validation of pathogen clearance studies should be performed in compliance with GLP (Good Laboratory Practices) guidance. Studies are conducted by using scale-down test systems representing the manufacturing process condition at proportional and process intermediates, the test articles artificially spiked with model viruses to evaluate viral clearance performance. The scale-down test systems are developed and qualified to represent the Current Good Manufacturing Practices (cGMP) process used in the manufacturing facilities. The viral clearance results, usually expressed as login virus reduction value (LRV), obtained from these small-scale studies, are representative of the viral clearance capacity of the corresponding process steps in the cGMP manufacturing facility.

So, the process development of each pathogen clearance step for a new therapeutic protein production requires significant effort and resources invested in wet laboratory experiments for process characterization studies.

### SUMMARY

In order to reduce such effort and resources, the present embodiments provide tools for predicting the performance of a (new) pathogen clearance process on the basis of past experiments.

In a first aspect, the present embodiments provide a computer-implemented method comprising:

receiving a multitude of training data sets
wherein each training data set of the multitude of training data sets comprises values of at least two process parameters and at least one value of a pathogen clearance score, the values of the process parameters characterizing a pathogen clearance process, and the value of the pathogen clearance score representing effectiveness and/or efficiency of the pathogen clearance process

building a pathogen clearance model on the basis of the multitude of training data sets
wherein the pathogen clearance model is configured to determine for a pathogen clearance process a value of a pathogen clearance score from values of process parameters characterizing the pathogen clearance process;

receiving an evaluation data set
wherein the evaluation data set comprises values of at least two process parameters, the values of the at least two process parameters characterizing a pathogen clearance process to be evaluated;

inputting the evaluation data set into the pathogen clearance model;

receiving as an output from the pathogen clearance model a resultant value of a pathogen clearance score; and

outputting the resultant value and/or one or more results related thereto.

In a second aspect, the present embodiments provide a computer system comprising:

a receiving unit

a processing unit, and

an output unit

wherein the receiving unit is configured to receive an evaluation data set, wherein the evaluation data set comprises values of at least two process parameters, the values of the at least two process parameters characterizing a pathogen clearance process to be evaluated, wherein the processing unit is further configured to input the evaluation data set into a pathogen clearance model and receive as an output from the pathogen clearance model a resultant value of a pathogen clearance score, the resultant value of the pathogen clearance score representing effectiveness and/or efficiency of the pathogen clearance process to be evaluated,

wherein the pathogen clearance model is configured on the basis of a multitude of training data sets to predict a relationship between process parameters of a pathogen clearance process and a pathogen clearance score, wherein the process parameters characterize the pathogen clearance process, and the pathogen clearance score represents effectiveness and/or efficiency of the pathogen clearance process,

wherein the output unit is configured to output the resultant value of the pathogen clearance score and/or one or more results related thereto.

In a third aspect, the present embodiments provide a non-transitory computer-readable storage medium comprising processor-executable instructions with which to perform an operation for determining a resultant value of a pathogen clearance score for a pathogen clearance process on the basis of an evaluation data set, the operation comprising:

receiving the evaluation data set, wherein the evaluation data set comprises at least two values of process parameters characterizing the pathogen clearance process;

determining from the evaluation data set the value of the pathogen clearance score by using a pathogen clearance model, wherein the pathogen clearance model is configured on the basis of a multitude of training data sets to predict a relationship between process parameters of a pathogen clearance process and a pathogen clearance score; and

outputting the resultant value and/or one or more results related thereto.

### DRAWINGS

The drawings described herein are for illustrative purposes only of selected embodiments and not all possible implementations and are not intended to limit the scope of the present disclosure.

FIG. 1 is a schematic of an exemplary purification process.

FIG. 2 is an example of a score plot obtained from a PCA analysis which provides an overview of performed experiments in a reduced dimensional space. Each data point in the score plot represents a single experiment. The score plot also helps in identifying groupings as shown by highlighted regions (Group-1 and Group-2).

FIG. 3 is an example of a contribution plot which enables the identification of process parameters responsible for the grouping of experimental runs.

FIG. 4 is an example of a loading plot which represents the relationship between score vectors in a reduced dimensional space and process parameters.

FIG. 5 is an example of a confusion matrix from which the accuracy of five different machine learning algorithms can be obtained: (A) OPLS-DA, (B) LR, (C) SVM, (D) DT, and (E) RF machine learning algorithm.

FIG. 6 is an example of a loading plot for an exemplary OPLS-DA classification model.

FIG. 7 is an example of a decision tree from an exemplary Random Forest classification model.

FIG. 8. is a schematic representation of a method of determination of process parameters for which certain criteria with respect to the pathogen clearance score are fulfilled.

FIG. 9 shows a graphical representation of ranges of temperature and pH to achieve a target LRV under parameter bounds as given in Table 2

FIG. 10 shows a comparison of measured vs. model predicted overall LRV of a Protein Chromatography OPLS regression model. Each data point in the plot represents a single experiment.

FIG. 11 shows a comparison of measured vs. model predicted protein recovery of a Protein-A chromatography OPLS regression model. Each data point in the plot represents a single experiment.

FIG. 12 is a block diagram of an exemplary computer system of the present disclosure suitable for determining one or more pathogen clearance score(s) for a pathogen clearance process on the basis of at least two process parameters.

### DETAILED DESCRIPTION

The embodiments will be more particularly elucidated below without distinguishing between the subjects of the embodiments (method, computer system, storage medium). On the contrary, the following elucidations are intended to apply analogously to all subjects of the embodiments, irrespective of in which context they occur.

The present embodiments serve to determine a pathogen clearance score for a pathogen clearance process.

A pathogen, in the broadest sense, is anything that can produce a disease and/or is able to do harm to a living organism. In some embodiments of the present invention, a pathogen is also referred to as an infectious microorganism or agent, such as a virus, bacterium, protozoan, prion, viroid, or fungus. In some embodiments, the pathogen is a virus.

In some embodiments of the present invention, the term “virus inactivation” is used as a synonym of the term “pathogen clearance”.

A pathogen clearance process is any activity which aims to remove a pathogen from a material, to reduce the amount of a pathogen in the material and/or to inactivate a pathogen, so that it does no harm any longer or less harm. In case of inactivation, pathogens may remain in the material, but in a non-infective or less infective form.

Typical methods for pathogen removal or for reducing the number of pathogens in a material include nanofiltration and chromatography. For details about nanofiltration and/or chromatography see the various literature related thereto (such as e.g. the chapter “Virus Removal by Nanofiltration” in the textbook: Therapeutic Proteins, edited by C. M. Smales and D. C. James, Springer Protocols 2005, pages 221-231; and the textbook: Downstream Industrial Biotechnology, edited by M. C. Flickinger, John Wiley & Sons, 2013, in particular chapters 9.7 to 9.9).

In particular, a pathogen clearance process commonly used in the production of therapeutic monoclonal antibodies is affinity chromatography purification. Affinity chromatography relies on the specific and reversible binding of antibodies to an immobilized ligand. A crude feed stock is passed through a column under conditions that promote binding of proteins (such as antibodies) in the feed stock to the ligands. After loading is complete, the column is washed under conditions that do not interrupt the specific interaction between the target protein and ligand, but that will disrupt any nonspecific interactions between process impurities (host cell proteins, etc.) and the stationary phase. The bound protein is then eluted with mobile phase conditions that disrupt the target/ligand interactions. The most applied affinity system for the purification of antibodies is the Staphylococcal protein A (Protein-A) and smaller ligands derived thereof. The affinity between Protein-A and IgG was one of the first native interactions to be explored for the development of an affinity system for protein purification. In addition to Protein-A, other immunoglobulin-binding bacterial proteins such as Protein-G, Protein-A/G and Protein-L are all commonly used to purify, immobilize or detect immunoglobulins. For more details about affinity chromatography see the various textbooks and articles related thereto, such as Affinity Chromatography—Methods and Protocols edited by P. Bailon et al., Methods in Molecular Biology Vol. 147, Humana Press Inc., 2000.

Many viruses contain lipid or protein coats that can be inactivated e.g. by chemical alteration or denaturation. Typical methods for such a pathogen inactivation include solvent/detergent inactivation, pasteurization (heating), acidic pH inactivation (also referred to as low pH inactivation), and ultraviolet inactivation. For details about pathogen inactivation see the various literature related thereto (such as e.g. Filtration and Purification in the Biopharmaceutical Industry, 3^{rd} edition, edited by M. W. Jornitz, CRC Press 2020; Continuous Biomanufacturing, edited by G. Subramanian, Wiley-VCH 2017).

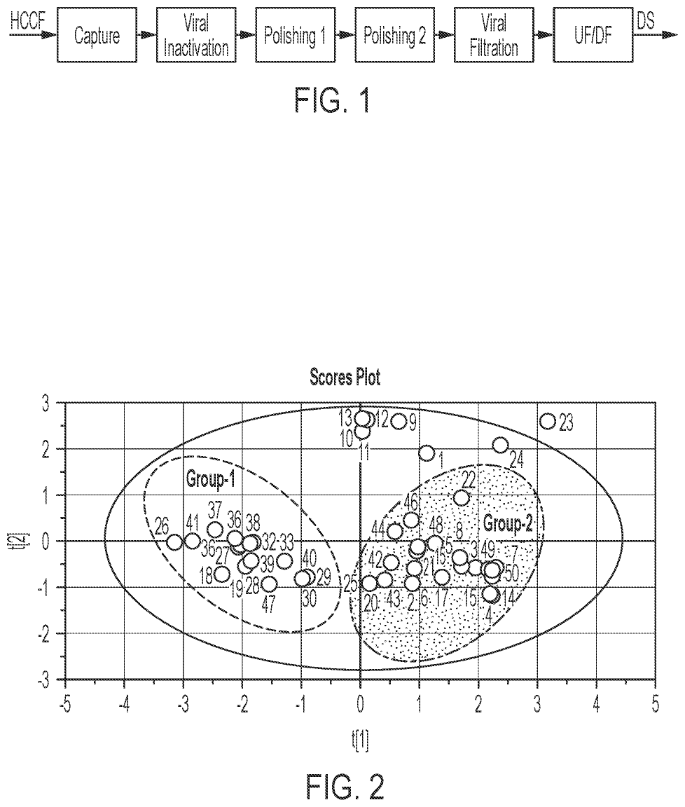

Often, different removal and/or inactivation steps are combined. A typical downstream purification process for a recombinant human monoclonal antibody (rhumAb) is shown in FIG. 1. The initial harvested cell culture fluid (HCCF) is first loaded onto a Protein-A column, then the captured rhumAb is eluted with a solution at low pH after thorough washes with equilibration and high salt wash buffers. Subsequently, the eluate is adjusted to low pH ranging from 3.7 to 3.9 and held for a duration time no less than two hours to inactivate enveloped viruses. The eluate post low pH viral inactivation is then neutralized and further polished by anion or cation exchange column or membrane adsorber chromatographic steps to remove impurities and viruses. The product intermediate will be further filtrated through viral filter to remove potential viruses.

In many cases, the concentration of viruses in a given sample is extremely low. In other extraction processes, low levels of impurity may be negligible, but because viruses are infective impurities, even one viral particle may be enough to ruin an entire process chain. Analytical limitations usually make it impossible to demonstrate absolute viral absence. Viral validation studies are, therefore, conducted both to document clearance of viruses known to be associated with the product, and to estimate the robustness of the process to clear potential adventitious viral contaminants (that may have gained access to the product) by characterizing the ability of the process to clear nonspecific “model” viruses.

A “spiking study” is a study done in order to determine the possible methods of viral removal or inactivation. For each process step to be evaluated for its virus inactivation/removal capacity, material is withdrawn from the previous manufacturing process step, a known amount of virus is added (spiking) and the sample is processed in a down-scaled version of the manufacturing process step. The amount of infectious virus before and after the down-scaled process step is measured e.g. by infecting indicator cells in an end-point-dilution set up. The virus reduction capacity of the process step can be calculated and presented e.g. as a logarithmic reduction value (LRV):

LRV=log₁₀[(V1×T1)/(V2×T2)]

wherein: V1=volume of spiked feedstock prior to the clearance step; T1=virus concentration of spiked feedstock prior to the clearance step; V2=volume of material after the clearance step; and T2=virus concentration of material after the clearance step.

The LRV is one example of a pathogen clearance score for a pathogen clearance process. The pathogen clearance score usually is a numerical value representing the effectiveness and/or efficiency of a pathogen clearance process.

According to the present embodiments, one or more pathogen clearance scores are determined using a pathogen clearance model which is built from a multitude of past experimental data. The past experimental data are also referred to herein as training data or training data set(s).

The past experimental data are a multitude of data sets. Each data set of the multitude of data sets comprises values of at least two process parameters and at least one value of a pathogen clearance score. The values of the at least two process parameters characterize a pathogen clearance process. The value of the pathogen clearance score represents effectiveness and/or efficiency of the respective pathogen clearance process.

The process parameters usually relate to the conditions under which the pathogen clearance process is/was performed.

If, for example, the pathogen clearance process is a low pH inactivation process, process parameters characterizing said process include e.g. temperature, pH value, incubation time, initial virus titer, sample volume, virus volume, spike ratio, type of protein (e.g. antibody class and/or subclass), and/or pH lowering agent. Further and/or other process parameters are conceivable.

If, for example, the pathogen clearance process is a Protein-A chromatography process (or any other affinity chromatography process), experimental features characterizing said process include e.g.
for the setup of the chromatography process: bed volume of the chromatography column, load capacity, loading density
for the equilibration phase: step volume, flow rate, conductivity, pH
for the load phase: step volume, flow rate, conductivity, spike dilution, protein concentration, pH, capacity
for the first washing phase: step volume, flow rate, conductivity, pH
for the second washing phase: step volume, flow rate, conductivity, pH
for the elution phase: step volume, flow rate, eluate pH, conductivity, protein concentration
for the regeneration phase: step volume, flow rate, conductivity, pH.

Further and/or other experimental features are conceivable.

The respective pathogen clearance score for the pathogen clearance process can e.g. be the LRV achieved in the pathogen clearance process, and/or any other value related to the performance (effectiveness and/or efficiency) of the pathogen clearance process.

The pathogen clearance score can e.g. be the time required to achieve a pre-defined concentration level of a pathogen in a material (inactivation time). In particular for a low pH inactivation process, in which the virus is subject to a low pH value for an incubation time, it can be interesting to know, how long the incubation time should last in order to completely inactivate viruses. Therefore, the inactivation time is a suitable pathogen clearance score for determining the performance of the pathogen clearance process.

In another preferred embodiment, the pathogen clearance score is the LRV or any other value related to the (residual) amount of pathogens in a material achieved after a fixed time period (e.g. after 30 minutes or 60 minutes or 90 minutes or 120 minutes or any other time period). Such a pathogen clearance score is particularly suitable for the assessment of the performance of a pathogen clearance process in which a material is subject to a certain treatment for a defined time period such as: low pH inactivation, heat treatment, UV irradiation and the like.

It is also possible to define classes, each class representing a value of the pathogen clearance score. To stay with the example of inactivation time: different pathogen clearance processes can be classified in accordance with the time it takes for complete virus inactivation. There can e.g. be two classes, a first class encompassing the pathogen clearance processes (characterized by their respective process parameters) in which complete inactivation was achieved within a pre-defined time limit, a second class encompassing the pathogen clearance processes in which the pathogens were not completely inactivated within the pre-defined time limit. The pathogen clearance score specifies to which class a certain pathogen clearance processes belongs to. The number of classes is of course not limited to two. It is possible to have more than two classes, e.g. three, four, five or more, each class representing a certain group of pathogen clearance processes having comparable (similar) performances.

In particular for affinity chromatography purification processes, the protein recovery (such as antibody recovery) is another example of pathogen clearance score.

From the multitude of (past, experimental) data sets a pathogen clearance model is built. Such a pathogen clearance model is a model which correlates process parameters of pathogen clearance processes with the respective pathogen clearance scores. There are numerous types of models and ways (algorithms) of creating those models, such as, but not limited to, random forest, support vector machine, logistic regression, tree based algorithms, naïve Bayes, linear/logistic regression, artificial neural networks, nearest neighbor methods, Gaussian process regression, and/or various forms of recommendation systems algorithms (for details, see e.g. Machine learning: a probabilistic perspective by Kevin P. Murphy, MIT press, 2012). The scores generated by various methods can be combined using methods such as, but not limited to, bagging and boosting, blending, ensemble methods, Bayesian model combination (BMC), simple averaging, weighted averaging, etc. (for details see e.g., Ensemble Methods in Data Mining: Improving Accuracy Through Combining Predictions by Giovanni Seni and John Elder, 2010 (Morgan and Claypool Publishers); Popular ensemble methods: An empirical study by Opitz & Maclin (1999), Journal of Artificial Intelligence Research 11: 169-98; and Ensemble-based classifiers by Rokach (2010), Artificial Intelligence Review 33 (1-2): 1-39).

Once the pathogen clearance model has been generated on the basis of the multitude of training data sets, the pathogen clearance model can be used to predict a pathogen clearance for a new pathogen clearance process.

The new pathogen clearance process is usually a pathogen clearance process which is subject to an evaluation. The aim of the evaluation is to determine the performance of the new pathogen clearance process (the new pathogen clearance process to be evaluated).

The pathogen clearance process to be evaluated is characterized by an evaluation data set. The evaluation data set comprises values of at least two process parameters, the values of the at least two process parameters characterizing the pathogen clearance process to be evaluated.

The evaluation data set is inputted into the pathogen clearance model. The pathogen clearance model outputs a resultant value of a pathogen clearance score. The resultant value of the pathogen clearance score is a numerical value representing the effectiveness and/or efficiency of the pathogen clearance process to be evaluated.

The resultant value of the pathogen clearance score of the pathogen clearance process to be evaluated can e.g. be outputted on a monitor and/or a printer. Instead of or in addition to outputting the resultant value, one or more results related to the resultant value can be outputted. The resultant value can e.g. be compared with a predefined reference value (e.g. a target pathogen clearance score). In case of a predefined deviation of the resultant value from the reference value, a message can be outputted, the message stating that the pathogen clearance process seems to be suitable for pathogen clearance or stating that the pathogen clearance process seems to be unsuitable for pathogen clearance.

A multitude of new pathogen clearance processes can be evaluated by determining their respective resultant values of a pathogen clearance score on the basis of the pathogen clearance model. The pathogen clearance processes of the multitude of new pathogen clearance processes can be ranked according to their resultant values. From that ranking list, a proportion of pathogen clearance processes (the top performers) can be selected for further evaluation (e.g. experimental validation of the prediction results).

So, the embodiments as described herein allow prioritization. It is not necessary to conduct a lot of experiments in order to determine whether a pathogen clearance process is able to remove and/or inactivate pathogens in a process in accordance with predefined performance criteria. It is possible to determine whether a new pathogen clearance process meets the performance criteria by calculating one or more pathogen clearance scores and comparing the one or more pathogen clearance score with one or more predefined reference values. It is possible to select the pathogen clearance processes for which the respective pathogen clearance scores meets predefined criteria. Selected pathogen clearance processes can be further evaluated experimentally; unselected pathogen clearance processes can be ignored.

Some examples of specific pathogen clearance models and pathogen clearance scores will be given hereinafter without the intention to limit the present embodiments to said examples.

In one embodiment of the present invention the pathogen clearance model is built by Principal Component Analysis.

Principal Component Analysis (PCA) is an unsupervised machine learning method used to reduce the dimensionality of data sets in which collinear relationships are present. As shown by way of example in Table 1, a model with four principal components captures a large majority (92%) of the variance present in a dataset.

TABLE 1
Summary of a Protein-A chromatography PCA model

| Statistic | Value |
|---|---|
| Number of Parameters | 31 |
| Number of Principal Components (PC) | 4 |
| R² (%) | 92 |
| Q² (%) | 83 |

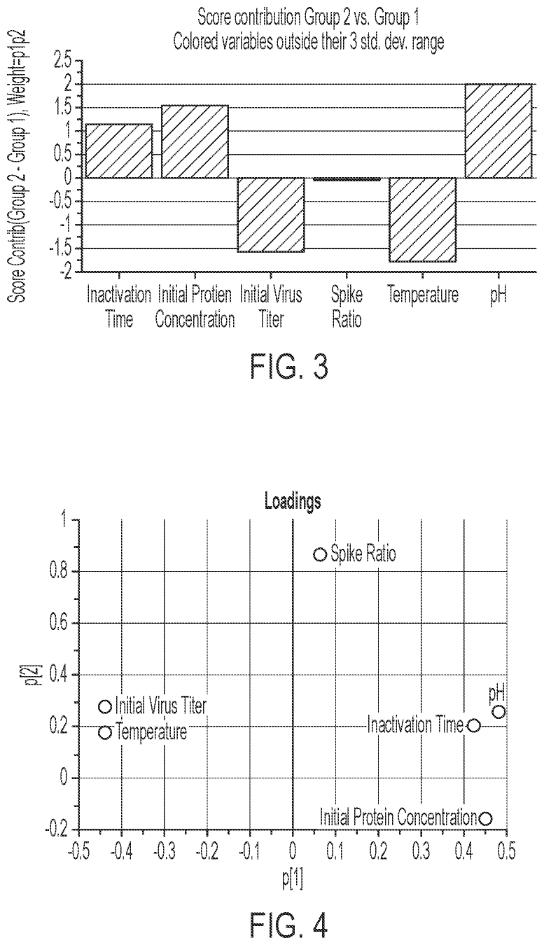

PCA provides an effective and efficient way of contextualizing experimental data. As shown in FIG. 2, a score plot can be used to identify groupings among experiments as well as atypical results. By color-coding the observations based on available meta data (e.g. product name), additional patterns could emerge. Moreover, PCA facilitates the identification of: (a) process parameters that drive groupings of the experimental results in the score plot, (b) relationship between process parameters. For example, differences between experimental results for low pH viral inactivation can be easily identified in FIG. 2 and linked to original process parameters via a contribution plot in FIG. 3. Also, the loading plot in FIG. 4 can reveal the relationships between the underlying process parameters and assist scientists to confirm known relationships or identify new ones. For instance, it is observed that inactivation time is positively correlated to pH.

In a preferred embodiment, the pathogen clearance model is trained using a supervised training method.

Supervised machine learning can be leveraged to model the relationship between process parameters and pathogen clearance performance. By way of example, a classification modeling approach was used to build a pathogen clearance prediction model for low pH virus inactivation. Two categories were defined based on the inactivation time:
1. fast inactivation (complete inactivation within certain time limit)
2. slow inactivation (incomplete inactivation within certain time limit).

The inactivation time is determined as the first time point where the virus titer drops below the assay limit of detection. This study assesses the predictive ability and interpretability of multiple machine learning algorithms. Specifically, the following algorithms were evaluated:
Orthogonal Partial Least Squares—Discriminant Analysis (OPLS-DA)
Logistic Regression (LR)
Support Vector Machine (SVM)
Decision Tree (DT)
Random Forest (RF)

The predictive ability of each classification model was evaluated using the overall model accuracy and individual class accuracy metrics calculated with cross-validation (CV). The CV overall accuracy and class accuracy were calculated as averages of their respective values over the total number of CV groups.

TABLE 2
Model performance summary for OPLS-DA, LR, SVM, DT, and RF

| | | | CV | | Test | |
|---|---|---|---|---|---|---|
| | | | Accuracy | Accuracy | Accuracy | Overall |
| | CV Overall | Overall | of Class | of Class | of Class | Test |
| | Accuracy | Accuracy | Slow | Slow | Slow | Accuracy |
| OPLS-DA | 0.89 | 0.89 | 0.92 | 0.93 | 1.00 | 1.00 |
| LR | 0.86 | 0.86 | 0.86 | 0.87 | 0.00 | 0.11 |
| SVM | 0.74 | 0.89 | 0.67 | 0.93 | 0.00 | 0.11 |
| DT | 0.83 | 1.00 | 0.86 | 1.00 | 0.00 | 0.11 |
| RF | 0.94 | 0.94 | 0.92 | 0.93 | 0.87 | 0.89 |

Based on the results for CV overall accuracy shown in Table 2, SVM and RF have the lowest (0.74) and highest (0.94) predictive ability, respectively. OPLS-DA with a CV overall accuracy of 0.89 outperforms LR, SVM, and DT with respective CV accuracies of 0.86, 0.74 and 0.83. The same conclusion can be drawn with regard to the accuracy for class “Slow”. RF is the only machine learning algorithm, among the ones evaluated in this work, which performed better than OPLS-DA. Although OPLS-DA has lower CV overall accuracy relative to RF, it still performed equally well for predicting class “Slow” with a CV class accuracy value of 0.92.

The overall accuracy of 1 clearly indicates the tendency of DT model to overfit. This is a condition where the model memorizes specific patterns in the training data set without being able to generalize them well to new data, as reflected in the lower overall accuracy score for cross-validation and low test accuracy score. Overfitting was addressed by using an ensemble of DT's in the Random Forest algorithm that resulted in superior predictive performance.

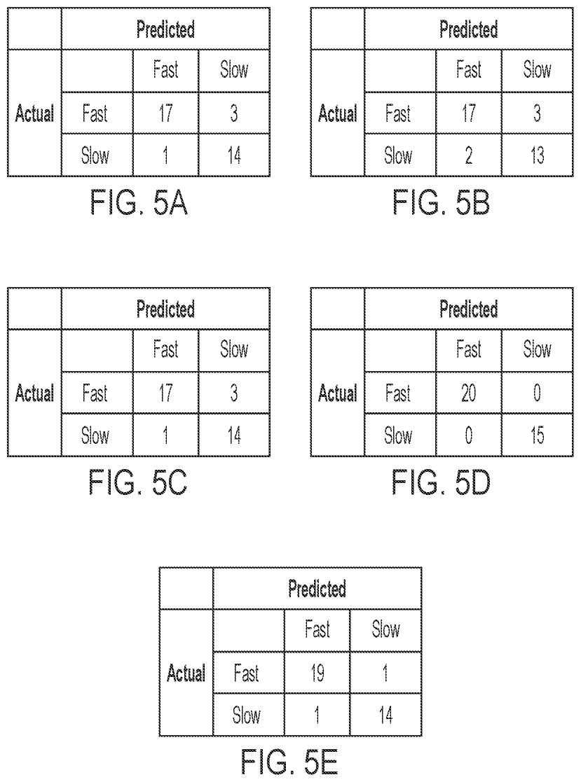

The confusion matrices were generated for all five machine learning algorithms, based on the entire training data set, as shown in FIG. 5. The confusion matrices were used to evaluate the performance of a classification model through the number of correctly and incorrectly predicted observations per class. For instance, there was only one incorrectly classified observation for each class for RF algorithm. However, for DT, there were none misclassified results for “Slow” and “Fast” classes which was earlier discussed as indicative of model overfitting. Thus, the use of cross-validation in conjunction with the confusion matrix can detect cases of model over-fitting enabling an effective assessment of model performance.

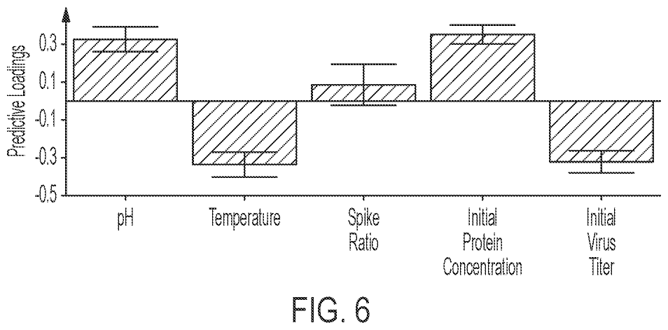

Each machine learning method provides different ways to interpret the modeling outcomes. In case of linear methods, a model can be interpreted in terms of direction and magnitude of the correlation between inputs and output. This is best demonstrated with the loading plot of the OPLS-DA model, shown in FIG. 6. For example, a positive loading for pH means that the higher the value of pH, the slower the inactivation.

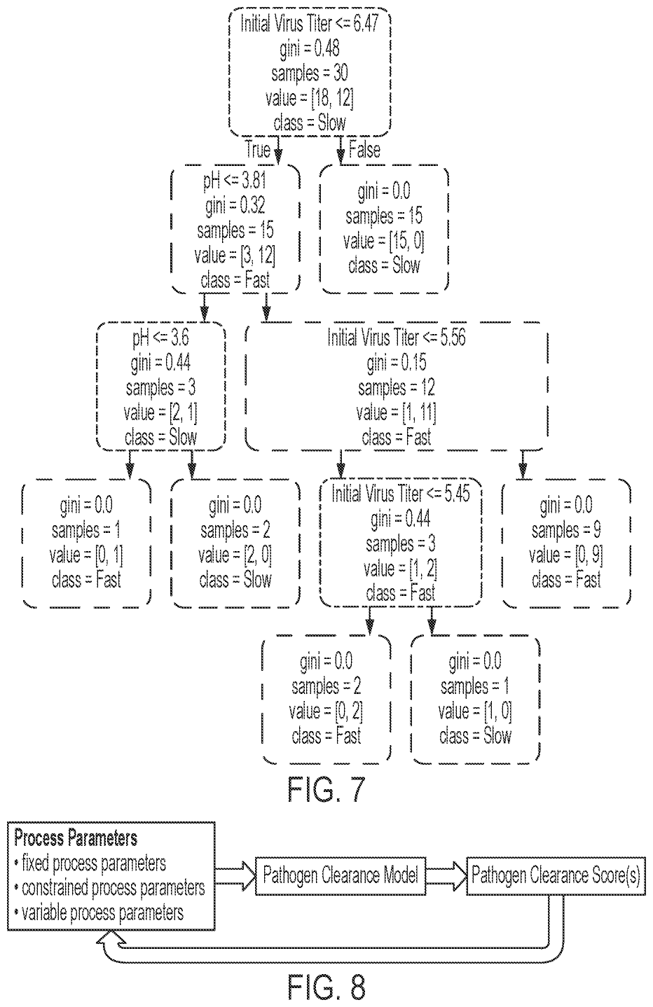

Nonlinear models may not be interpreted in the same way as linear ones. Tree-based classification models maximize their accuracies by finding split(s) in predictor variable(s) where the Gini index is minimized. As shown in FIG. 7, one of the decision trees from the Random Forest classification model identifies that Initial Virus Titer and pH are two most important variables in achieving high model accuracy.

If a pathogen clearance model is built, it can also be used for the determination of process ranges for which pre-defined requirements for the pathogen clearance score are met. In a first step, a target pathogen clearance score is defined, e.g. a threshold which should not be transgressed. Then combinations of process parameters can be determined for which the respective pathogen clearance score does not transgress the pre-defined threshold. A schematic representation of the method is given in FIG. 8. To allow determination of process parameters that satisfy a given pathogen clearance score, the inverse problem of mapping a pathogen clearance score to process parameters needs to be solved. This inverse problem is formulated as a constrained optimization problem the numerical solution of which yields the combinations of process parameters that satisfy a target pathogen clearance score. For such an optimization problem, one or more of the process parameters could be fixed or subject to certain constraints. This is illustrated by the following example of a low pH virus inactivation process. The set-up for the example is outlined in Table 3.

TABLE 3
Operating bounds on time-series model parameters

| Parameter | Bounds or fixed value |
|---|---|
| pH | [3.6, 4.05] |
| Temperature (° C.) | [14, 21] |
| Incubation Time (min) | 120 |
| Spike Ratio | 20 |
| Initial Protein Concentration (mg/ml) | 15 |
| Initial Virus Titer | 5.74 |
| Target LRV | ≥5.00 |

The target LRV was defined to be 5.00 with defined bounds for the process parameters pH and temperature. All other process parameters and conditions were kept fixed.

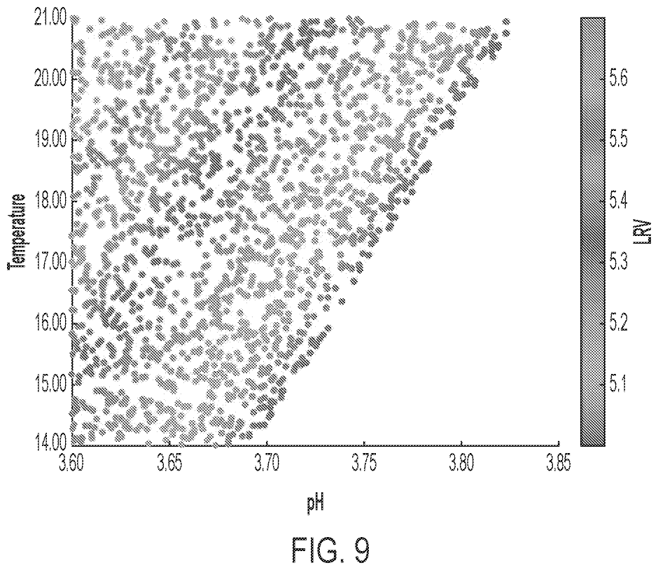

The outcome is shown in FIG. 9. Each point plotted in the figure corresponds to a valid solution which satisfies the target LRV and the pre-defined constraints. The region where a combination of process parameters satisfies the target LRV is displayed by data points colored by the achieved LRV. If the target LRV cannot be achieved with a particular combination, no point is plotted. For instance, at a pH of 3.65, for all temperatures, an LRV of at least 5 can be achieved. This is not the case for higher pH values, for a pH of 3.8, a temperature of >19.5° C. needs to be used to achieve the target LRV. This application can allow the evaluation of how changes in requirements on process parameters translate into achievable LRV.

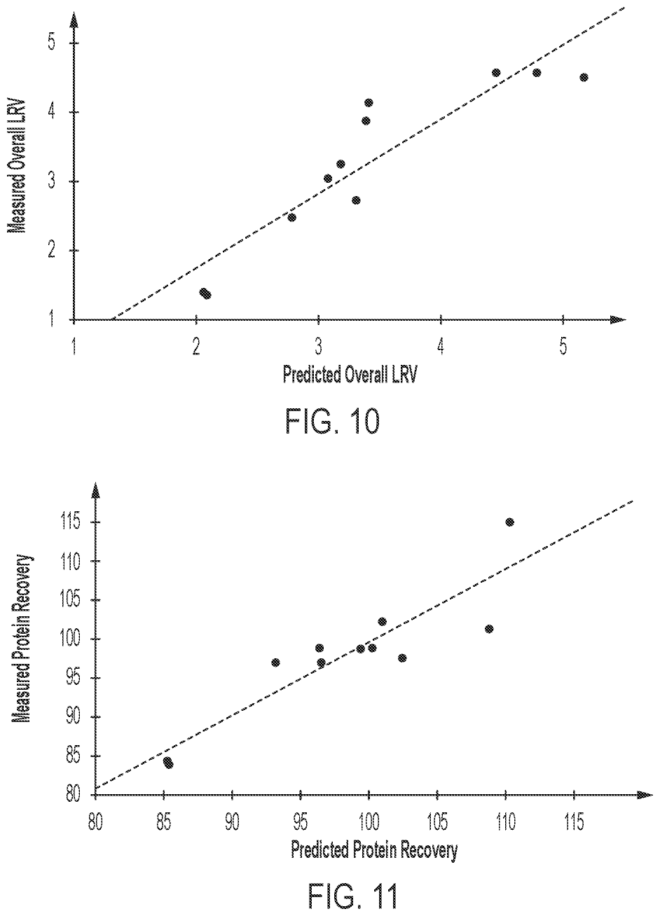

FIG. 10 and FIG. 11 show the prediction accuracy of a Protein-A chromatography OPLS (Orthogonal Partial Least Squares) regression model. The model was used to predict the LRV as well as protein recovery of an overall Protein-A chromatography process. The process parameters are summarized in Table 4. Some of the process parameters were non-linearly transformed in order to take account of their non-linear relation to LRV and protein recovery.

TABLE 4
Input variables and their transformations
for a Protein-A chromatography model

| Input Variable | Input Variable Transformation |
|---|---|
| Setup Loading Density | None |
| Setup Scale Factor | None |
| Setup Load Capacity | None |
| Equilibration Conductivity | None |
| Equilibration pH | Exponential |
| Load Protein Concentration | None |
| Load Sample Virus Titer | None |
| Load Capacity | None |
| 1^{st} Washing Phase: Conductivity | None |
| 2^{nd} Washing Phase: Conductivity | None |
| 1^{st} Washing Phase: pH | Exponential |
| 2^{nd} Washing Phase: pH | Exponential |
| Eluate pH | Exponential |
| Elution Conductivity | None |

Another example of a pathogen clearance model is an artificial neural network that is trained to determine one or more pathogen clearance scores from values of process parameters.

Such an artificial neural network comprises at least three layers of processing elements: a first layer with input neurons (nodes), an Nth layer with at least one output neuron (node), and N−2 inner layers, where N is a natural number greater than 2. In such a network, the output neuron(s) serve(s) to predict at least one value of at least one pathogen clearance score. The input neurons serve to receive values of process parameters. The processing elements of the layers are interconnected in a predetermined pattern with predetermined connection weights therebetween. Each network node represents a simple calculation of the weighted sum of inputs from prior nodes and a non-linear output function. The combined calculation of the network nodes relates the inputs to the output(s). Separate networks can be developed for each property measurement or groups of properties can be included in a single network.

Training estimates network weights that allow the network to calculate (an) output value(s) close to the measured output value(s). A supervised training method can be used in which the output data is used to direct the training of the network weights. The network weights are initialized with small random values or with the weights of a prior partially trained network. The training data inputs are applied to the network and the output values are calculated for each training sample. The network output values are compared to the measured output values. A backpropagation algorithm is applied to correct the weight values in directions that reduce the error between measured and calculated outputs. The process is iterated until no further reduction in error can be made or until a predefined prediction accuracy has been reached. A cross-validation method can be employed to split the data into training and validation data sets. The training data set is used in the backpropagation training of the network weights. The validation data set is used to verify that the trained network generalizes to make good predictions. The best network weight set can be taken as the one that best predicts the outputs of the test data set. Similarly, varying the number of network hidden nodes and determining the network that performs best with the data sets optimizes the number of hidden nodes.

Forward prediction uses the trained network to calculate one or more pathogen clearance score(s) for a (new) process on the basis of its process parameters. Values of the process parameters are inputted into the trained network. A feed forward calculation through the network is made to predict the output property value(s). The predicted measurements can be compared to (a) property target value(s) or tolerance(s). Since the method of the embodiments is based on historical data of property values, the prediction of property values using such method typically have an error approaching the error of the empirical data, so that the predictions are often just as accurate as verification experiments.

Details of setting up an artificial neural network and training the network can be found e.g. in C. C. Aggarwal: Neural Networks and Deep Learning, Springer 2018, ISBN 978-3-319-94462-3.

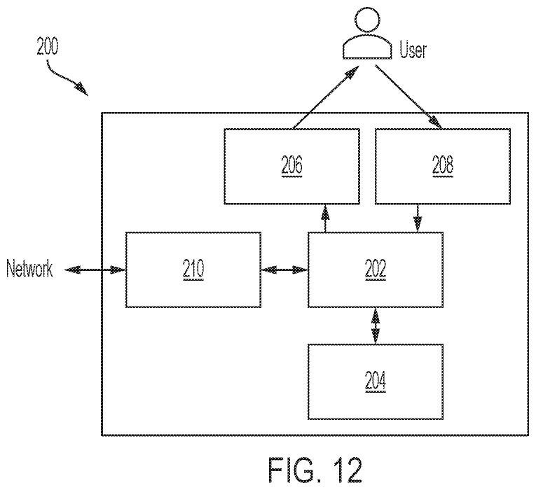

The present embodiments are carried out by using a computer system. FIG. 12 illustrates an exemplary computer system 200. In connection therewith, the computer system 200 may be configured, by executable instructions, to implement the various algorithms and other operations described herein.

The exemplary computer system 200 may include, for example, one or more servers, workstations, personal computers, laptops, tablets, smartphones, other suitable computing devices, combinations thereof, etc. In addition, the computer system 200 may include a single computing device, or it may include multiple computing devices located in close proximity or distributed over a geographic region, and coupled to one another via one or more networks.

Such networks may include, without limitations, the Internet, an intranet, a private or public local area network (LAN), wide area network (WAN), mobile network, telecommunication networks, combinations thereof, or other suitable network(s), etc.

With that said, the illustrated computer system 200 includes a processing unit 202 and a memory 204 that is coupled to (and in communication with) the processing unit 202. The processing unit 202 may include, without limitation, one or more processors (e.g., in a multi-core configuration, etc.), including a central processing unit (CPU), a microcontroller, a reduced instruction set computer (RISC) processor, an application specific integrated circuit (ASIC), a programmable logic device (PLD), a gate array, and/or any other circuit or processor capable of the functions described herein. The above listing is exemplary only, and thus is not intended to limit in any way the definition and/or meaning of the term processing unit.

The memory 204, as described herein, is one or more devices that enable information, such as executable instructions and/or other data, to be stored and retrieved. The memory 204 may include one or more computer-readable storage media, such as, without limitation, dynamic random access memory (DRAM), static random access memory (SRAM), read only memory (ROM), erasable programmable read only memory (EPROM), solid state devices, flash drives, CD-ROMs, thumb drives, tapes, hard disks, and/or any other type of volatile or nonvolatile physical or tangible computer-readable media. The memory 204 may be configured to store, without limitation, process parameters, pathogen clearance scores, pathogen clearance models and/or other types of data (and/or data structures) suitable for use as described herein, etc. In various embodiments, computer-executable instructions may be stored in the memory 204 for execution by the processing unit 202 to cause the processing unit 202 to perform one or more of the functions described herein, such that the memory 204 is a physical, tangible, and non-transitory computer-readable storage media. It should be appreciated that the memory 204 may include a variety of different memories, each implemented in one or more of the functions or processes described herein.

In the exemplary embodiment, the computer system 200 also includes an output unit 206 that is coupled to (and is in communication with) the processing unit 202. The output unit 206 outputs, or presents, to a user of the computer system 200, by, for example, displaying and/or otherwise outputting information such as, but not limited to, pathogen clearance score(s), process parameters, and/or any other type of data. It should be further appreciated that, in some embodiments, the output unit 206 may comprise a display device such that various interfaces (e.g., applications (network-based or otherwise), etc.) may be displayed at computer system 200, and in particular at the display device, to display such information and data, etc. And in some examples, the computer system 200 may cause the interfaces to be displayed at a display device of another computing device, including, for example, a server hosting a website having multiple webpages, or interacting with a web application employed at the other computing device, etc. Output unit 206 may include, without limitation, a liquid crystal display (LCD), a light-emitting diode (LED) display, an organic LED (OLED) display, an “electronic ink” display, combinations thereof, etc. In some embodiments, the output unit 206 may include multiple units.

The computer system 200 further includes an input device 208 that receives input from a user. The input device 208 is coupled to (and is in communication with) the processing unit 202 and may include, for example, a keyboard, a pointing device, a mouse, a stylus, a touch sensitive panel (e.g., a touch pad or a touch screen, etc.), another computing device, and/or an audio input device. Further, in some exemplary embodiments, a touch screen, such as that included in a tablet or similar device, may perform as both the output unit 206 and the input device 208. In at least one exemplary embodiment, the output unit 206 and the input device 208 may be omitted.

In addition, the illustrated computer system 200 includes a network interface 210 coupled to (and in communication with) the processing unit 202 (and, in some embodiments, to the memory 204 as well). The network interface 210 may include, without limitation, a wired network adapter, a wireless network adapter, a telecommunications adapter, or other device capable of communicating to one or more different networks.

The network interface (210) and/or the input device (208) are also referred to as receiving unit herein.

1. A computer-implemented method comprising:
(a) receiving a multitude of training data sets, wherein each training data set of the multitude of training data sets comprises values of at least two process parameters and at least one value of a pathogen clearance score, the values of the process parameters characterizing a pathogen clearance process, and the value of the pathogen clearance score representing effectiveness and efficiency of the pathogen clearance process;
(b) building a pathogen clearance model on the basis of the multitude of training data sets wherein the pathogen clearance model is configured to determine for a pathogen clearance process a value of a pathogen clearance score from values of process parameters characterizing the pathogen clearance process;
(c) receiving an evaluation data set, wherein the evaluation data set comprises values of at least two process parameters, the values of the at least two process parameters characterizing a pathogen clearance process to be evaluated,
(d) inputting the evaluation data set into the pathogen clearance model; and
(e) receiving as an output from the pathogen clearance model a resultant value of a pathogen clearance score outputting the resultant value and one or more results related thereto.

2. The computer-implemented method according to claim 1, wherein the pathogen clearance process is a low pH virus inactivation process.

3. The computer-implemented method according to claim 2, wherein the pathogen clearance score is a virus reduction capacity in the form of logarithmic reduction value after a pre-defined time period and inactivation time required to achieve a pre-defined concentration level of viruses in a material.

4. The computer-implemented method according to claim 3, wherein the process parameters are two or more parameters selected from the group consisting of: temperature, pH value, incubation time, initial virus titer, sample volume, virus volume, spike ratio, type of protein, and a lowering agent.

5. The computer-implemented method according to claim 1, wherein the pathogen clearance process is an affinity chromatography process used for purification of antibodies.

6. The computer-implemented method according to claim 5, wherein the pathogen clearance score is a virus reduction capacity in the form of a logarithmic reduction value and an amount of recovered antibodies.

7. The computer-implemented method according to claim 6, wherein process parameters are two or more parameters selected from each of the groups consisting of: for setup of the chromatography process: bed volume of the chromatography column, load capacity, loading density; for equilibration phase: step volume, flow rate, conductivity, pH; for load phase: step volume, flow rate, conductivity, spike dilution, protein concentration, pH, capacity; for first washing phase: step volume, flow rate, conductivity, pH; for second washing phase: step volume, flow rate, conductivity, pH; for elution phase: step volume, flow rate, eluate pH, conductivity, protein concentration; and for regeneration phase: step volume, flow rate, conductivity, and pH.

8. The computer-implemented method according to claim 1, wherein the pathogen clearance model is selected from the group consisting of: orthogonal partial least squares discriminant analysis model, logistic regression model, support vector machine, random forest, gradient boosting, and artificial neural network.

9. The computer-implemented method according to claim 1, further comprising: receiving a target pathogen clearance score; receiving information about process parameter constraints;
determining combinations of resultant values of process parameters which fulfill the process parameter constraints and achieve the target pathogen clearance score; and outputting the values of the resultant values of process parameters.

10. The computer-implemented method according to claim 1, further comprising:
determining a multitude of resultant values for a multitude of pathogen clearance processes to be evaluated; ranking the pathogen clearance processes of the multitude of pathogen clearance processes according to their resultant values, thereby generating a ranking list;
selecting from the ranking list a proportion of top performers; and conducting experiments with selected top performers for validation purposes.

11. A computer system, comprising:
(a) a receiving unit;
(b) a processing unit; and
(c) an output unit. wherein the receiving unit is configured to receive an evaluation data set, wherein the evaluation data set comprises values of at least two process parameters, the values of the at least two process parameters characterizing a pathogen clearance process to be evaluated,
wherein the processing unit is further configured to input the evaluation data set into a pathogen clearance model and receive as an output from the pathogen clearance model a resultant value of a pathogen clearance score, the resultant value of the pathogen clearance score representing effectiveness and efficiency of the pathogen clearance process to be evaluated,
wherein the pathogen clearance model is configured on the basis of a multitude of training data sets to predict a relationship between process parameters of a pathogen clearance process and a pathogen clearance score, wherein the process parameters characterize the pathogen clearance process, and the pathogen clearance score represents effectiveness and efficiency of the pathogen clearance process, and
wherein the output unit is configured to output the resultant value of the pathogen clearance score and one or more results related thereto.

12. A non-transitory computer-readable storage medium comprising processor-executable instructions with which to perform an operation for determining a resultant value of a pathogen clearance score for a pathogen clearance process on the basis of an evaluation data set, the operation comprising:
(a) receiving the evaluation data set, wherein the evaluation data set comprises at least two values of process parameters characterizing the pathogen clearance process;
(b) determining from the evaluation data set the value of the pathogen clearance score by using a pathogen clearance model, wherein the pathogen clearance model is configured on the basis of a multitude of training data sets to predict a relationship between process parameters of a pathogen clearance process and a pathogen clearance score; and
(c) outputting the resultant value and one or more results related thereto.
